(19) United States
(12) Patent Application Publication
(10) Pub. No.: US 2023/0320609 A1
(43) Pub. Date: Oct. 12, 2023

(54) WIRELESS, INTERNET-BASED SYSTEM FOR MONITORING LYMPHEDEMA

(71) Applicants: Baxter International Inc., Deerfield, IL (US); Baxter Healthcare SA, Glattpark (Opfikon) (CH)

(72) Inventors: Matthew Banet, San Diego, CA (US); Zachary Plona, Baltimore, MD (US); Lina Ngao, Baltimore, MD (US); Hunter Hutchinson, Baltimore, MD (US); Benjamin Monteagudo, Baltimore, MD (US); Jennifer Schultz, Baltimore, MD (US); Ignacio Albert Smet, San Diego, CA (US)

(21) Appl. No.: 18/296,241

(22) Filed: Apr. 5, 2023

Related U.S. Application Data
(60) Provisional application No. 63/327,956, filed on Apr. 6, 2022.

(51) Int. Cl. A61B 5/0537; A61B 5/257; A61B 5/28; A61B 5/296; A61B 5/00

(52) U.S. Cl. CPC A61B 5/0537; A61B 5/257; A61B 5/28; A61B 5/296; A61B 5/4872

(57) ABSTRACT

The invention provides a system for characterizing fluids in a tissue located in a portion of a patient. The system features an impedance system that includes a current-injecting electrode that injects an electrical current into the portion of the patient and a signal-measuring electrode that measures an impedance signal affected by the injected electrical current and an amount of the fluids. At least one of the electrodes includes an alignment feature that, during use, is aligned on the portion using a marking on the portion. The system also includes a processing system that receives the impedance signal from the impedance system or a signal determined from it. It then processes the signal to determine a parameter related to the degree of fluids in the tissue. The marking, for example, can be a permanent or semi-permanent marking, such as a tattoo.

### PRIORITY CLAIM AND CROSS-REFERENCE TO RELATED APPLICATIONS

This application claims priority to and the benefit of U.S. Provisional Patent App. No. 63/327,956 filed Apr. 6, 2022, titled WIRELESS, INTERNET-BASED SYSTEM FOR MONITORING LYMPHEDEMA, the entire contents of which are incorporated by reference herein in their entirety and relied upon.

### FIELD OF THE INVENTION

The invention described herein relates to systems for monitoring patients, specifically those suffering from lymphedema, in both hospital and home environments.

### BACKGROUND

Unless a term is expressly defined herein using the phrase “herein “______””, or a similar sentence, there is no intent to limit the meaning of that term beyond its plain or ordinary meaning. To the extent that any term is referred to in this document in a manner consistent with a single meaning, that is done for the sake of clarity only; it is not intended that such claim term be limited to that single meaning. Finally, unless a claim element is defined by reciting the word “means” and a function without the recital of any structure, it is not intended that the scope of any claim element be interpreted based on the application of 35 U.S.C. § 112(f).

The lymphatic system is a network of vessels primarily responsible for the collection and distribution of interstitial fluid throughout the body. The fluid that the lymphatic vessels carry—lymph—consisting of cellular waste products, proteins, peptides, and immune cells. When this vascular network is compromised, it is unable to drain and distribute fluid to the appropriate parts of the body. Consequently, unwanted lymphatic fluid buildup can lead to increased infection risk and severe swelling, giving rise to the condition known as lymphedema.

More specifically, lymphedema is a disease characterized by the accumulation of extracellular fluid or a buildup of adipose tissue in the interstitium due to reduced lymphatic transport in the setting of normal capillary filtration. Lymphedema is a chronic, incurable, progressive, and potentially debilitating condition, with numerous studies demonstrating notably reduced quality of life outcomes for those with chronic limb changes (Ahmed, R. L., et al. (2008). Lymphedema and quality of life in breast cancer survivors: the Iowa Women's Health Study. Journal of Clinical Oncology, 26(35): p. 5689.; Penha, T. R. L., et al. (2016). Quality of Life in Patients with Breast Cancer—Related Lymphedema and Reconstructive Breast Surgery. Journal of reconstructive microsurgery, 32(06): p. 484-490.). Globally, lymphatic filariasis, a parasitic disease, causes lymphedema in over 120 million people in 72 countries. Development of lymphedema following a surgery or other medical intervention is commonly referred to as ‘secondary lymphedema.’ Although secondary lymphedema may arise due to infectious causes or cardiac, vascular, and renal impairment, most commonly secondary lymphedema develops after cancer surgery to remove lymph nodes, such as axillary lymph node dissection (herein “ALND”) in the breast cancer population. Depending on the number of lymph nodes removed, a patient's risk of lymphedema following ALND may be as high as 50% (Cemal, Y., Pusic, A., and Mehrara, B. (2011). Preventative Measures for Lymphedema: Separating Fact from Fiction. Journal of the American College of Surgeons, 213(4), 543-551. https://doi.org/10.1016/j.jamcollsurg.2011.07.001.). Furthermore, research in the last decade has demonstrated that the development of lymphedema is not limited to the breast cancer population alone. Lymphedema has been shown to be a well-known complication following treatment of melanoma, gynecologic cancer, genitourinary cancer, head, and neck cancer, sarcoma, radiation therapy, and pelvic dissection, with incidence ranging from 4-31% (Cormier, J. N., et al. (2010). Lymphedema beyond breast cancer. Cancer, 116, 5138-5149. https://doi.org/10.1002/cncr.25458.). While patients are at risk of developing lymphedema at any point in their life following cancer treatment, it is estimated that 80% of secondary lymphedema cases develop within the first two years following treatment and that 89% of all cases develop within the first three years following treatment (Norman, S. A., et al. (2009). Lymphedema in breast cancer survivors: incidence, degree, time course, treatment, and symptoms. Journal of Clinical Oncology, 27(3): p. 390.).

The progression of lymphedema can be slowed or arrested using complete decongestive therapy (herein “CDT”), which typically consists of physiotherapy, compression, massage, and other techniques. Early intervention has been shown to have both outcomes and cost benefits (Shah, C., et al. (2016). The impact of early detection and intervention of breast cancer-related lymphedema: A systematic review. Cancer Medicine, 5(6), 1154-1162. https://doi.org/10.1002/cam4.691/.). Furthermore, current National Comprehensive Cancer Network (herein “NCCN”) guidelines on breast cancer recognize the importance of lymphedema as a possible complication of axillary lymph node surgery and recommend early diagnosis and intervention for lymphedema (National Comprehensive Cancer Network. Breast Cancer (Version 7.2021). https://www.nccn.org/professionals/physician_gls/pdf/breast.pdf. Accessed Aug. 26, 2021.). For patients with breast cancer-related lymphedema, it has been estimated that without documented intervention, 48% of mild cases progress to the late stage of the disease, where limb changes become irreversible (Ad, V. B., et al. (2010). Time course of mild arm lymphedema after breast conservation treatment for early-stage breast cancer. International Journal of Radiation Oncology* Biology* Physics, 76(1): p. 85-90.).

Clinicians commonly rely on patient-reported symptoms of limb changes such as heaviness, tingling, or aching as the first sign that lymphedema may be present. The disease is then typically diagnosed using clinical exams and limb volume measurement or estimation. Common limb volume assessment methods include segmental circumference measurement, water volume displacement, and perometry, which uses infrared optical sensors to estimate limb volume. Complex radiologic imaging may also be performed to stage disease progressions, such as lymphoscintigraphy, magnetic resonance imaging, and computerized tomography. More recently, bioimpedance is an effective method of early diagnosis of lymphedema (Warren, A., Janz, B., Slavin, S. & Borud, L. (2007). The Use of Bioimpedance Analysis to Evaluate Lymphedema. Annals of Plastic Surgery, 58(5), 541-543. doi:10.1097/01.sap.0000244977.84130.cf.).

Bioimpedance analyzes tissue body-worn electrodes passing an alternating electrical current through a portion of the body to measure the impedance of current flow. This measurement technique operates on the principle that different tissues have different properties of resistance. For example, fat and bone act as insulators, while electrolytic fluids conduct electricity. In general, high-frequency currents pass through intracellular (herein “ICF”) and extracellular fluid (herein “ECF”), while low frequency currents are only able to pass through extracellular fluid. In this way, the impedance of intra- and extracellular fluid can be measured using the principle:

R_{TOTAL}=R_{ECF}+R_{ICF}

Where R_{TOTAL} is the impedance measured through a tissue when a high-frequency current is applied, and R_{ECF} is the impedance measured through a tissue when a low-frequency current is applied. Subtracting R_{ECF} from R_{TOTAL} yields R_{ICF}, which is the impedance corresponding to intracellular tissue. Since lymphatic fluid conducts electricity and accumulates in the extracellular space, its presence can be characterized by a drop in bioimpedance, particularly R_{ECF}.

Wearable bioimpedance sensors have been designed for cardiac indications (see, for example, U.S. Pat. No. 9,913,612, the contents of which are incorporated herein by reference). A primary example of this technology is Baxter's CoVa 2 Monitoring System, which has been FDA-cleared to measure cardiac output, stroke volume, chest fluid, heart rate, and other variables. CoVa 2 is a necklace-shaped device that attaches to a patient's chest with a pair of disposable electrodes. Using bioimpedance measures the parameters mentioned above and wirelessly transmits digitized numerical values and time-dependent waveforms to the cloud, where clinicians analyze them to diagnose the patient. Systems like these allow for patient monitoring in the home, which reduces the need for additional clinic visits. A wearable solution such as this could bring great value in the context of the lymphedema patient population, as these patients often have limited mobility and face a high number of clinic visits for oncologic and lymphedema treatment.

Despite significant work in the fields of lymphedema and fluid monitoring, there remains a need for a lymphedema-detection device that would allow patients or providers to take tissue property measurements quickly, reliably, easily, reproducibly, and quantitatively. Early detection of lymphedema allows for more prompt initiation of low-cost preventative therapies, leading to improved clinical outcomes and decreased cost to the patient and the healthcare system. There is a pressing need for a convenient and accurate method to detect lymphedema early in the disease progression, particularly before its progression to the second disease stage, when tissue changes become permanent. As there is no cure for lymphedema, the late-stage disease can only be managed using complete decongestive therapy, compression garments, and in some instances, microvascular surgical intervention—all these therapies at a high cost to the patient (Boyages J, et al. (2017). Financial cost of lymphedema borne by women with breast cancer. Psychooncology, 26(6):849-855. doi: 10.1002/pon.4239. Epub 2016 Aug. 21. PMID: 27479170.). Furthermore, without comprehensive knowledge about the fluid and fat composition of a patient's affected limb, the most appropriate surgical treatment cannot be prescribed. The lack of such a measurement for lymphedema progression limits the invention and adoption of novel treatment options which require clinical evidence. The only commercially available bioimpedance solution on the market, the SOZO device by Impedimed (see, for example, U.S. Pat. No. 10,653,334), is expensive, limited to in-office use only, and requires significant patient mobility to place hands and feet on the device for a measurement to occur.

In view of the foregoing, it would be beneficial to improve upon conventional approaches for generally measuring swelling and, more specifically, lymphedema in patients located in both the hospital and the home.

### SUMMARY OF THE INVENTION

Given the above, in one aspect, the invention provides a system for characterizing fluids in a tissue located in a portion of a patient. The system features an impedance system that includes a current-injecting electrode that injects an electrical current into the portion of the patient, and a signal-measuring electrode that measures an impedance signal affected by the injected electrical current and an amount of the fluids. At least one of the electrodes includes an alignment feature that, during use, is aligned on the portion using a marking on the portion. The system also includes a processing system that receives the impedance signal from the impedance system, or a signal determined from it, and then processes the signal to determine a parameter related to the degree of fluids in the tissue.

The marking, for example, can be a permanent or semi-permanent marking, such as a tattoo. The tattoo can be a conventional tattoo used for medical purposes (e.g., radiation therapy for cancer), or alternatively a tattoo such as that described by Ephemeral Tattoo that has a limited lifetime, e.g., about one year. Alternately the tattoo could be comprised of a conductive material which matches to an electrical component of the alignment feature.

In embodiments, the alignment feature is one of an opening, optically transparent area, notch, or cut-out area configured to be disposed above the marking. The alignment feature is configured so that the marking can be visualized when the electrode containing the alignment feature is attached to the portion. In related embodiments, the alignment feature is a component configured to contact the patient proximal to the marking.

Typically, an external component that is remote from the patient includes the processing system, and is typically a hand-held component, e.g., a component mounted on the back surface of a mobile phone. It typically connects to the electrodes through a cable. In this and other embodiments, the hand-held component includes a wireless component, e.g., a Bluetooth® transceiver, a Wi-Fi transceiver, or a near field communication (herein “NFC”) tag from an NFC component. In related embodiments, the wireless transceiver may be a cellular modem operating on a cellular network that connects directly to the cloud. Typically, the hand-held component connects through the wireless or other means to an Internet-based component that receives information from the hand-held component. For example, the Internet-based component may be a system operating in the cloud (e.g., through Amazon Web Services) and features a web-based system with interfaces for both clinicians and the patient. The hand-held component may also include a mobile application that integrates to the Internet-based component.

In other embodiments, the processing system is worn directly on the patient's body. Here, the processing system is typically attached to the patient's body by one of the electrodes. In either case, the processing system typically operates a computer code that processes the impedance signal to determine the parameter related to the degree of fluids in the tissue. For example, the computer code can process a DC impedance signal (e.g., a change in this signal) to generate an index related to the degree of fluid build-up in the patient, e.g., the degree of lymphedema.

In preferred embodiments, the impedance system features two current-injecting electrodes and two signal-measuring electrodes. These electrodes typically include a hydrogel conductor that is matched to the electrical and mechanical impedance of the patient's skin. To this end, the impedance system typically features two electrode patches, with each electrode patch comprising one current-injecting electrode and one signal-measuring electrode.

In embodiments, the system features an electrical cable that connects the two electrode patches. For example, the electrical cable can be a stretchable cable, e.g., one that includes a conductor characterized by a resistance that changes with mechanical strain. Here, the conductor is typically coupled to a strain gauge. In related embodiments, the electrical conductor is in electrical contact with a distance-measuring component that generates a signal related to a degree that the stretchable cable stretches. For example, the distance-measuring component can include a capacitor characterized by a capacitance value that changes with the degree that the stretchable cable stretches. Alternatively, the distance-measuring component includes a resistor characterized by a resistance value that changes with the degree that the stretchable cable stretches. And in still other embodiments, the distance-measuring component is a piezoelectric material that generates a voltage that changes with the degree that the stretchable cable stretches.

In other embodiments, the system additionally includes an optical system featuring a light source and a photodetector. The light source is typically configured to irradiate the tissue located in the portion of a patient, and the photodetector is configured to detect radiation from the light source after it irradiates the tissue and, in response, generate a radiation-induced signal. Here, the processing system receives the radiation-induced signal, or a signal determined from it, and process it along with the impedance signal or a signal determined from it to determine the parameter related to the degree of fluids in the tissue in the portion of the patient. In related embodiments, the optical system features multiple light sources. For example, light sources of different wavelengths to examine the composition of the tissue and degree of fluids in the portion of a patient, e.g., amount of hemoglobin and deoxyhemoglobin. As another example, the light sources may be located at varying locations from the photodetector to obtain radiation-induced signals at multiple locations and/or depths in the portion of a patient. Alternatively, the optical system features multiple photodetectors. As an example, photodetectors at varying locations from a light source may generate radiation-induced signals at multiple locations and/or depths in the tissue in the portion of a patient. And in still other embodiments, the optical system features multiple light sources and photodetectors to obtain a combination of the above features.

In other embodiments, the system includes an electrocardiogram (herein “ECG”) system in electrical contact with the signal-measuring electrode that measures an ECG waveform. The processing system can process the ECG waveform to determine a value of heart rate (herein “HR”) corresponding to the patient.

In another aspect, the invention provides a system for characterizing fluids in a tissue located in a portion of a patient. The system includes an impedance system featuring at least one current-injecting electrode configured to inject an electrical current into the portion of the patient, and one signal-measuring electrode configured to measure an impedance signal affected by the electrical current and the fluids. The system also includes a distance-measuring system featuring a stretchable cable connected on one distal end to the current-injecting electrode, and at an opposing distal end to the signal-measuring electrode. The distance-measuring system generates an electrical signal that varies with a degree that the stretchable cable is stretched. The system also includes a processing system that receives the impedance signal from the impedance system and the electrical signal from the distance-measuring system, or signals determined therefrom, and collectively process them to determine an index related to an amount of fluids in the tissue in the portion of the patient.

In yet another aspect, the invention provides a system for characterizing fluids in a tissue located in a portion of a patient that includes an impedance measurement system similar to that described above, and a strain-measuring system comprising an electrical conductor coupled to the portion of the patient. The electrical conductor is characterized by an electrical resistance signal that varies with strain imparted on the electrical conductor by the fluids. A processing system receives the impedance signal from the impedance system and the electrical resistance signal from the strain-measuring system, or signals determined from these, and collectively processes them to determine an index related to the amount of fluids in the tissue in the portion of the patient.

And it yet another aspect, the invention contains any of the above-described systems embedded in a wearable cuff or sleeve. Such systems can also include methods of inflation (e.g., a pump) that serve two purposes: they force sensors within the cuff or sleeve to contact the patient's body and can also provide therapeutic compression to ameliorate the impact of lymphedema. Furthermore, the embedded sensors could be a combination of pressure sensors, e.g., piezoresistive or piezoelectric, or strain gauges.

In other embodiments, the system includes an electromyography (herein “EMG”) sensor, consisting of two measuring electrodes and a reference electrode, which measures electrical signals from muscle activation. Such EMG sensors can be used to evaluate swelling, inflammation, necrosis, and limb fat content.

In other embodiments, the circuit system also includes a motion sensor, such as an accelerometer or gyroscope. And in still other embodiments, the circuit system additionally consists of a flash memory system that stores digital representations of the signals. In other embodiments, the invention contains any of the above-described sensors embedded in a wearable ring for a finger or toe. For example, to measure an ECG or impedance waveform waveforms, the sensor would include an inner set of electrodes that contact a finger on the hand that the ring is worn and an outer set of electrodes that are contacted with fingers from the opposite hand. In a related embodiment, the invention includes any of the above-described systems embedded in a pair of glasses or spectacles. Here, for example, the sensors could be located on the pads for the bridge of the nose and behind the ears. In other embodiments, one of the sensors could be a flexible ultrasound system in a patch that adheres directly to the skin. Such a system could include electrodes made from polyimide, Cu, Cu/Sn, piezoelectric materials (e.g., pillars), and similar materials.

In light of the disclosure set forth herein, and without limiting the disclosure in any way, in a first aspect of the present disclosure, which may be combined with any other aspect, or portion thereof, a system for characterizing fluids in a tissue located in a portion of a patient includes an impedance system and a processing system. The impedance system includes at least one current-injecting electrode configured to inject an electrical current into the portion of the patient, and at least one signal-measuring electrode configured to measure an impedance signal affected by the injected electrical current and an amount of the fluids. The system includes an alignment feature that during use is aligned on the portion using a marking on the portion. The processing system is configured to receive the impedance signal from the impedance system, or a signal determined therefrom, and then process it to determine a parameter related to the degree of fluids in the tissue.

In a second aspect of the present disclosure, which may be combined with any other aspect, or portion thereof, the alignment feature is one of an opening, optically transparent area, notch, or cut-out area configured to be disposed above the marking.

In a third aspect of the present disclosure, which may be combined with any other aspect, or portion thereof, the opening, optically transparent area, notch, or cut-out area is configured to allow the marking to be visualized when at least one of the current-injecting electrode and the signal-measuring electrode is attached to the portion.

In a fourth aspect of the present disclosure, which may be combined with any other aspect, or portion thereof, the alignment feature is a component configured to contact the patient proximal to the marking.

In a fifth aspect of the present disclosure, which may be combined with any other aspect, or portion thereof, the processing system is comprised by an external component that is remote from the patient.

In a sixth aspect of the present disclosure, which may be combined with any other aspect, or portion thereof, the external component connects to at least one of the current-injecting electrode and the signal-measuring electrode through a cable.

In a seventh aspect of the present disclosure, which may be combined with any other aspect, or portion thereof, the external component is a hand-held component.

In an eighth aspect of the present disclosure, which may be combined with any other aspect, or portion thereof, the hand-held component comprises a wireless component.

In a ninth aspect of the present disclosure, which may be combined with any other aspect, or portion thereof, the wireless component comprises one of a Bluetooth® transceiver or a Wi-Fi transceiver.

In a tenth aspect of the present disclosure, which may be combined with any other aspect, or portion thereof, the system further includes an Internet-based component configured to receive information from the hand-held component.

In an eleventh aspect of the present disclosure, which may be combined with any other aspect, or portion thereof, the processing system is configured to be worn on the patient's body.

In a twelfth aspect of the present disclosure, which may be combined with any other aspect, or portion thereof, the processing system is attached to the patient's body by one of the current-injecting electrode and the signal-measuring electrode.

In a thirteenth aspect of the present disclosure, which may be combined with any other aspect, or portion thereof, the processing system operates a computer code that processes the impedance signal to determine the parameter related to the degree of fluids in the tissue.

In a fourteenth aspect of the present disclosure, which may be combined with any other aspect, or portion thereof, the computer code is configured to process a DC impedance signal.

In a fifteenth aspect of the present disclosure, which may be combined with any other aspect, or portion thereof, the computer code is configured to process a change in the DC impedance signal.

In a sixteenth aspect of the present disclosure, which may be combined with any other aspect, or portion thereof, the parameter is an index related to the change in the DC impedance signal.

In a seventeenth aspect of the present disclosure, which may be combined with any other aspect, or portion thereof, the parameter indicates a degree of lymphedema in the patient.

In an eighteenth aspect of the present disclosure, which may be combined with any other aspect, or portion thereof, the impedance system comprises two current-injecting electrodes and two signal-measuring electrodes.

In a nineteenth aspect of the present disclosure, which may be combined with any other aspect, or portion thereof, the impedance system comprises two electrode patches, with each electrode patch comprising one current-injecting electrode and one signal-measuring electrode.

In a twentieth aspect of the present disclosure, which may be combined with any other aspect, or portion thereof, the system further includes an electrical cable connecting the two electrode patches.

In a twenty-first aspect of the present disclosure, which may be combined with any other aspect, or portion thereof, the electrical cable is a stretchable cable.

In a twenty-second aspect of the present disclosure, which may be combined with any other aspect, or portion thereof, the electrical cable further comprises a conductor characterized by a resistance that changes with mechanical strain.

In a twenty-third aspect of the present disclosure, which may be combined with any other aspect, or portion thereof, the conductor is comprised by a strain gauge.

In a twenty-fourth aspect of the present disclosure, which may be combined with any other aspect, or portion thereof, the electrical conductor is in electrical contact with a distance-measuring component that generates a signal related to a degree that the stretchable cable stretches.

In a twenty-fifth aspect of the present disclosure, which may be combined with any other aspect, or portion thereof, the distance-measuring component comprises a capacitor characterized by a capacitance value that changes with the degree that the stretchable cable stretches.

In a twenty-sixth aspect of the present disclosure, which may be combined with any other aspect, or portion thereof, the distance-measuring component comprises a resistor characterized by a resistance value that changes with the degree that the stretchable cable stretches.

In a twenty-seventh aspect of the present disclosure, which may be combined with any other aspect, or portion thereof, the distance-measuring component is a piezoelectric or piezoresistive material that generates a voltage that changes with the degree that the stretchable cable stretches.

In a twenty-eighth aspect of the present disclosure, which may be combined with any other aspect, or portion thereof, the system further includes an optical system comprising a light source and a photodetector.

In a twenty-ninth aspect of the present disclosure, which may be combined with any other aspect, or portion thereof, the light source is configured to irradiate the tissue located in the portion of a patient.

In a thirtieth aspect of the present disclosure, which may be combined with any other aspect, or portion thereof, the photodetector is configured to detect radiation from the light source after it irradiates the tissue and, in response, generate a radiation-induced signal.

In a thirty-first aspect of the present disclosure, which may be combined with any other aspect, or portion thereof, the processing system is further configured to receive the radiation-induced signal, or a signal determined therefrom, and process it along with the impedance signal or a signal determined therefrom to determine the parameter related to the degree of fluids in the tissue in the portion of the patient.

In a thirty-second aspect of the present disclosure, which may be combined with any other aspect, or portion thereof, the system further includes an ECG system in electrical contact with the signal-measuring electrode and configured to measure an ECG waveform.

In a thirty-third aspect of the present disclosure, which may be combined with any other aspect, or portion thereof, the processing system is further configured to process the ECG waveform to determine a heart rate corresponding to the patient.

In a thirty-fourth aspect of the present disclosure, which may be combined with any other aspect, or portion thereof, the system further includes an EMG system in electrical contact with the signal-measuring electrode and configured to measure EMG signals from muscle neurons in the patient.

In a thirty-fifth aspect of the present disclosure, which may be combined with any other aspect, or portion thereof, the processing system is further configured to process the EMG signals to determine adipose tissue concentration corresponding to the patient.

In a thirty-sixth aspect of the present disclosure, which may be combined with any other aspect, or portion thereof, the electrodes are embedded in a wearable cuff that is worn on an arm of the patient.

In a thirty-seventh aspect of the present disclosure, which may be combined with any other aspect, or portion thereof, the system further includes a cuff comprising an inflatable mechanism that inflates the cuff and pushes the electrodes against the skin.

In a thirty-eighth aspect of the present disclosure, which may be combined with any other aspect, or portion thereof, a system for characterizing fluids in a tissue located in a portion of a patient includes an impedance system, a distance-measuring system, and a processing system. The impedance system includes at least one current-injecting electrode configured to inject an electrical current into the portion of the patient, and at least one signal-measuring electrode configured to measure an impedance signal affected by the electrical current and the fluids. The distance-measuring system includes a stretchable cable connected on one distal end to the at least one current-injecting electrode and at an opposing distal end to the at least one signal-measuring electrode. The distance-measuring system is configured to generate an electrical signal that varies with a degree that the stretchable cable is stretched. The processing system is configured to receive the impedance signal from the impedance system and the electrical signal from the distance-measuring system, or signals determined therefrom, and collectively process them to determine an index related to an amount of fluids in the tissue in the portion of the patient.

In a thirty-ninth aspect of the present disclosure, which may be combined with any other aspect, or portion thereof, a system for characterizing fluids in a tissue located in a portion of a patient includes an impedance system, a strain-measuring system, and a processing system. The impedance system includes at least one current-injecting electrode configured to inject an electrical current into the portion of the patient, and at least one signal-measuring electrode configured to measure an impedance signal affected by the electrical current and the fluids. The strain-measuring system includes an electrical conductor coupled to the portion of the patient, the electrical conductor characterized by an electrical resistance signal that varies with strain imparted on the electrical conductor by the fluids. The processing system is configured to receive the impedance signal from the impedance system and the electrical resistance signal from the strain-measuring system, or signals determined therefrom, and collectively process them to determine an index related to an amount of fluids in the tissue in the portion of the patient.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1A-B are drawings showing front views of, respectively, legs and arms corresponding to healthy limbs (on the left-hand side) and limbs with a severe case of lymphedema (on the right-hand side);

FIG. 2 is a schematic drawing of the system according to the invention featuring a sensor attached to a patient suffering from lymphedema that sends information through a mobile device to a cloud-based system;

FIGS. 3A-E are schematic drawings showing side views of a portion of a patient impacted by lymphedema being measured by, respectively, an impedance sensor, a strain gauge, a temperature sensor, an optical sensor, and a vibration sensor;

FIG. 4A is a graph showing changes in impedance measured as a function of fluid volume using an impedance sensor similar to that shown in FIG. 3A;

FIGS. 4B and 4C are graphs showing changes in impedance measured using an impedance sensor similar to that shown in FIG. 3A from, respectively, a sample containing an insulating fluid and a conducting fluid;

FIGS. 5A and 5B are graphs showing changes in impedance, measured at different frequencies of injected current, as a function of fluid volume using an impedance sensor similar to that shown in FIG. 3A from, respectively, a sample containing an insulating fluid and a conducting fluid;

FIG. 5C is a schematic drawing of relatively high and low-frequency currents injected during an impedance measurement that pass, respectively, through cells and around cells in human tissue;

FIG. 6A is a schematic drawing of the sensor according to the invention attached to a patient;

FIGS. 6B and 6C are schematic drawings of, respectively, top and bottom surfaces of electrodes used by the sensor of FIG. 6A to measure impedance from the patient;

FIGS. 6D and 6E are schematic drawings of, respectively, the sensor shown in FIG. 6A detached and attached to a patient with semi-permanent markings used to align the disposable electrodes;

FIG. 7A is an image of a printed circuit board used in the hand-held component of the system of FIG. 2;

FIG. 7B is a photograph of the printed circuit board shown in FIG. 7A;

FIG. 8A is a schematic drawing of an electrode patch used in an embodiment of the invention;

FIG. 8B is a schematic drawing of a bottom surface of a circuit board used in a device that connects to the electrode patch shown in FIG. 8A during measurement of lymphedema;

FIG. 8C is a schematic drawing of an enclosure that encloses the circuit board of FIG. 8C;

FIGS. 9A and 9B are graphs of time-dependent waveforms featuring heartbeat-induced pulses measured from a patient's chest using, respectively, an ECG sensor and an impedance sensor within the system of FIG. 1A;

FIG. 10 is a flow chart of an algorithm used by the system of FIG. 1A to estimate the degree of a patient's lymphedema;

FIGS. 11A and 11B are schematic drawings of an alternate, a patch-based embodiment of the invention attached, respectively, to an arm and foot of a patient suffering from lymphedema;

FIG. 11C is a schematic drawing of the patch-based embodiment of the invention shown in both FIGS. 11A and 11B;

FIG. 11D is a schematic drawing of the patch-based embodiment of the invention shown in FIG. 12C that sends information through a mobile device to a cloud-based system;

FIG. 12A is a schematic drawing of an alternate, patch-based embodiment of the invention attached to an arm of a patient suffering from lymphedema, which may be utilized in conjunction with injection of a lymphatic contrast agent or dye;

FIG. 12B is a schematic drawing of the bottom surface of the patch-based embodiment of the invention shown in FIG. 12A;

FIG. 12C is a schematic drawing of the top surface of the patch embodiment shown in FIG. 12A;

FIG. 12D is a schematic drawing of a system similar to that shown in FIG. 12A, but with multiple patches applied on the limb;

FIG. 12E is a schematic drawing of a system similar to that shown in FIG. 12A, but wherein the patch is held in place with a strap as opposed to an adhesive

FIGS. 13A and 13B are schematic drawings of, respectively, a cuff-based and sleeve-based embodiment of the invention worn on an arm by a patient suffering from lymphedema;

FIG. 14 is a schematic drawing of a patch-based embodiment of the invention that uses EMG sensors instead of bioimpedance sensors;

FIGS. 15A and 15B are, respectively, schematic drawings of markings on the patient's skin before and after lymphedema-induced swelling; and

FIG. 16 is a schematic drawing of an ultrasound sensor used to measure lymphedema-induced swelling in a patient's arm.

### DETAILED DESCRIPTION

Although the following text sets forth a detailed description of numerous different embodiments, the legal scope of the invention described herein is defined by the words of the claims set forth at the end of this patent. The detailed description is to be construed as exemplary only; it does not describe every possible embodiment, as this would be impractical, if not impossible. One ordinary skill in the art could implement numerous alternate embodiments, which would still fall within the scope of the claims.

### 1. SYSTEM OVERVIEW

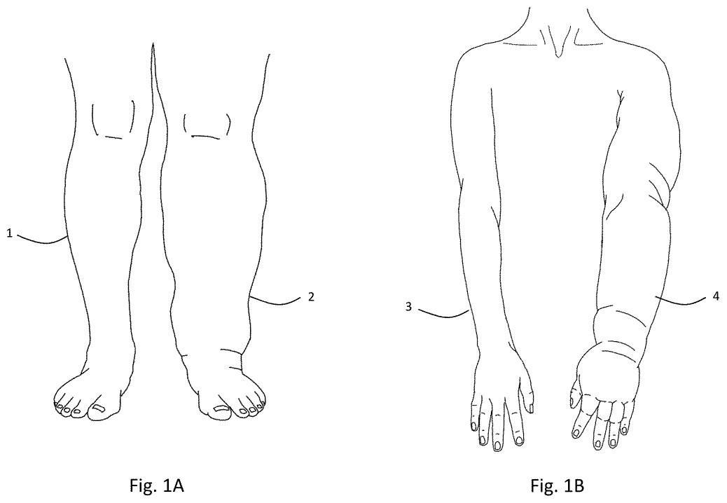

Referring to FIGS. 1A-B and 2, lymphedema, as described is above, is a condition causing swelling in portions of a patient, such as the leg 2, as shown in FIG. 1A, or the arm 3 as shown in FIG. 1B. Typically, lymphedema-induced swelling increases gradually; over time, this causes, for example, a normal leg 1 to evolve to a slightly swollen leg, and ultimately to a profoundly swollen leg 2. Similarly, a normal arm 3 can evolve to a profoundly swollen arm 4. At this point, the condition can be quite painful and, in some cases, irreversible.

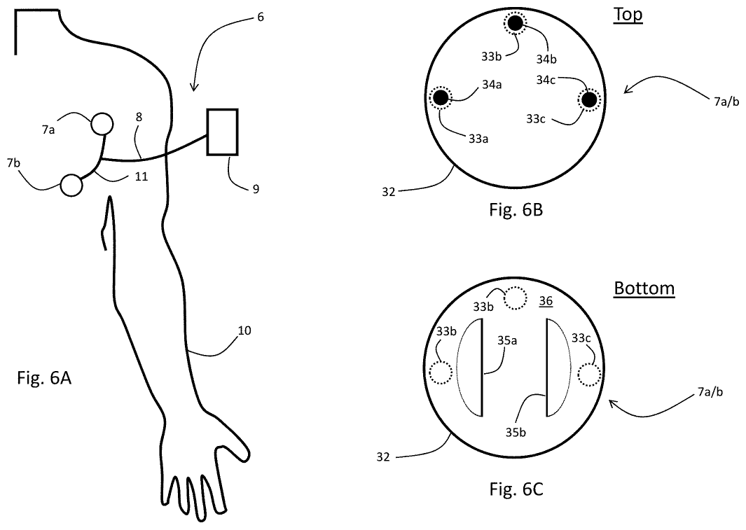

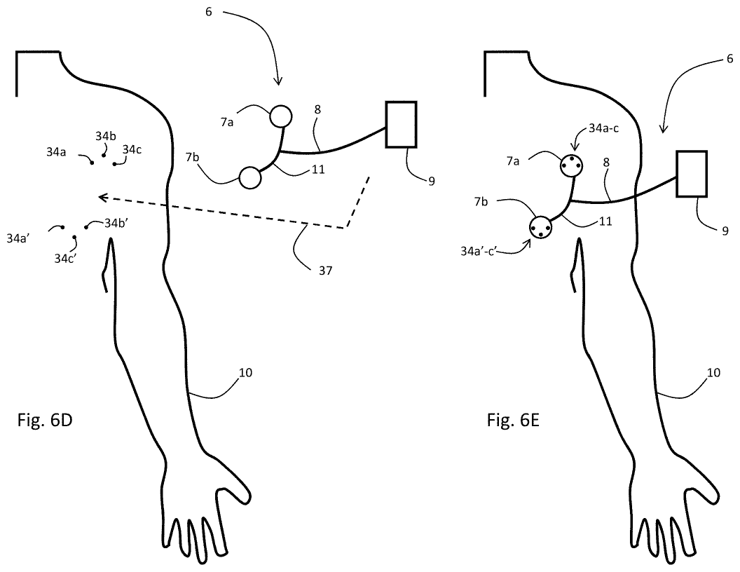

To monitor this gradual swelling in the chest 5 (or any other portion of the body, such as the leg 2 and arm 4 in FIGS. 1A-B, respectively), the invention provides a system 6 featuring a pair of body-worn electrode patches 7a,b that adhere to the region of interest and connect through a cable 8 to a hand-held component 9. Because lymphedema is typically a slowly varying condition—often manifesting over several months—system 6 is generally only used once or twice a month and often for only several minutes. During typical use, the patient applies the body-worn electrode patches 7a,b to the region of interest and then disposes of them after use. To ensure they are placed in the same location for each use—a requirement for consistent measurements of impedance and strain, which in turn will vary with lymphedema-induced swelling—the patches 7a,b typically include an alignment feature, such as an optically transparent area, notch, or cut-out area configured to be disposed above a semi-permanent marking on the patient 10, as shown in FIGS. 6B-E, 12B-C, 13B-C, and 15A. The semi-permanent marking, for example, can be a temporary tattoo, such as a tattoo that fades away after about 1 year, as developed by the company Ephemeral Tattoo (https://ephemeral.tattoo/how-it-works/). Referring to FIGS. 15A-B, the tattoo 101a may include components (in this case, vertical lines) separated by a known distance that separate because of lymphedema, essentially resulting in a modified tattoo 101b as the unswollen limb 102a evolves into a swollen limb 102b. This separation can be identified visually 103, with a camera 105, or with tape 107.

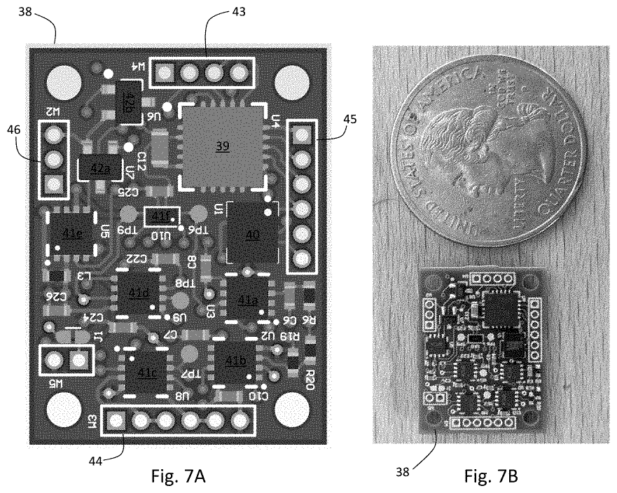

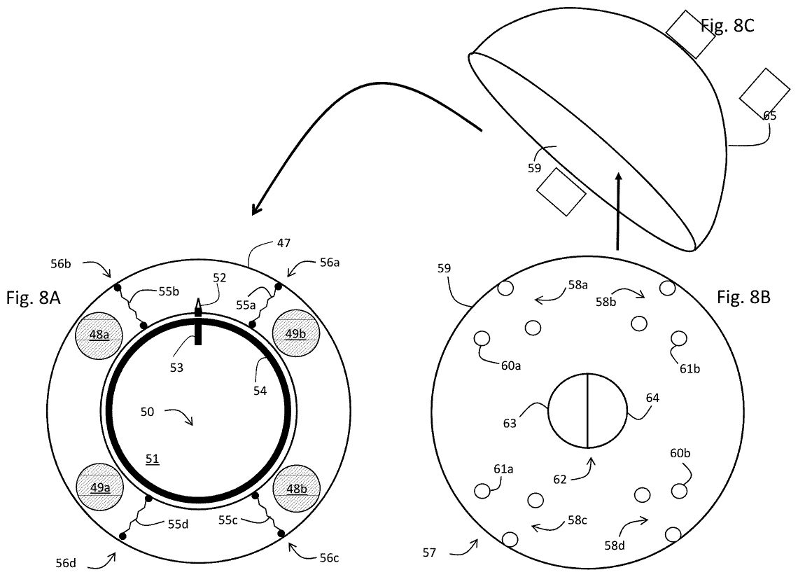

The hand-held component 9 encloses a printed circuit board, such as that shown in FIGS. 7A-B, which features circuitry for making impedance and strain measurements. For the impedance measurement, each body-worn electrode patch 7a,b includes a current-injecting electrode and a sensing electrode (shown, for example, in FIG. 6C). Both electrodes typically feature a hydrogel pad that sticks to the patient and conducts current from the current-injecting electrode into the patient and bio-electric signals from the patient into the cable 8. During a measurement, the current-injecting electrode typically injects high-frequency (e.g., 100 kHz), low-amperage (e.g., 4 mA) current into the portion of the patient impacted by lymphedema; the current and amount of underlying fluid impacts the bio-electric signals sensed by the sensing electrode. The body-worn electrode patches 7a,b are typically separated by a few centimeters on the patient and connect with a stretchable cable 11 that features an electrical conductor characterized by electrical properties that vary with the degree to which the cable 11 is stretched. Typically, the electrical conductor features a coiled metal strand with an electrical resistance that increases as it is stretched. Alternatively, the conductor may be separated into two unique conductors that sandwich an insulating material on opposing sides, thus effectively forming a variable capacitor. Here, as the cable 11 stretches, the internal conductors on the opposing surface are drawn closer together, thus decreasing the inherent capacitance of the system. In related embodiments, the cable can also include a piezoelectric material that generates a voltage that varies with the amount that it is stretched. In these cases, changes in the electrical properties of the cable 11—either resistance, capacitance, or voltage—can be easily detected using circuitry within the printed circuit board. Typically, this is done by including a resistor of known resistance in-line with the variable in-cable resistor or a capacitor of known capacitance in-line with the variable in-cable capacitor and then monitoring a voltage drop across the different circuit elements. An internal analog-to-digital converter digitizes the voltage drop. It converts it into a digital DC signal, which can then be processed with an algorithm described below. Similarly, the analog-to-digital converter can measure voltages generated by the piezoelectric material, which indicates the degree of stretching. Algorithms for processing these signals are encoded on a microprocessor on the printed circuit board within the hand-held component 9 and typically feature a series of processing steps as described by the flow chart in FIG. 11.

As lymphedema worsens in severity, the portion of tissue underneath the body-worn electrode patches 7a,b fills with fluid and swells. The increase in underlying fluid causes the resistance and impedance of the portion to change; the impedance circuit measures this. Concurrently the swelling increases the distance between the body-worn electrode patches 7a,b, which in turn increases the degree of stretching of the stretchable cable 11. This changes its resistance and/or capacitance, or the voltage it generates, as described above. The algorithm processes the digitized DC signals—which represent changes in impedance, resistance, capacitance, and/or voltage—to calculate a parameter related to the severity of the patient's lymphedema. A wireless transceiver within the hand-held device 9, e.g., a Bluetooth® or Wi-Fi transmitter, sends a digitized version of the parameter to a matched transceiver the patient's mobile phone 13, as indicated by the arrow 12. The mobile phone 13 then forwards the digitized version of the parameter to a cloud-based system 15, as indicated by the arrow 14. Typically, the cloud-based system 15 renders the parameter for both patients and clinicians or forwards the parameter (e.g., through a web services interface) to a third-party software application, such as a mobile application.

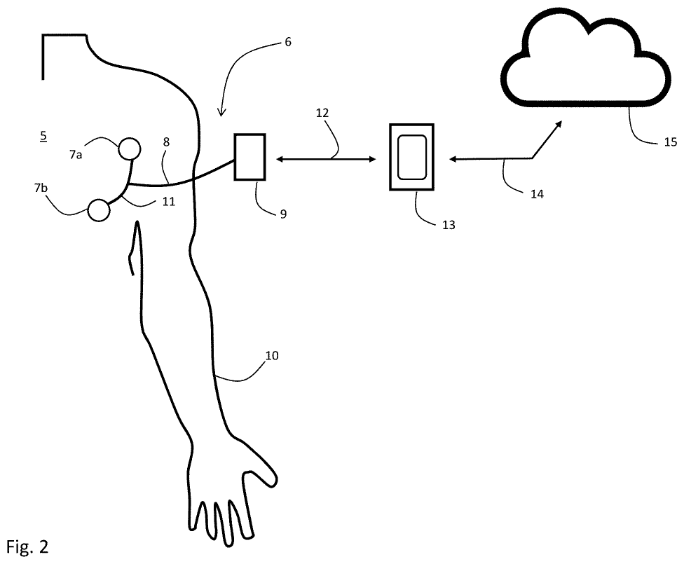

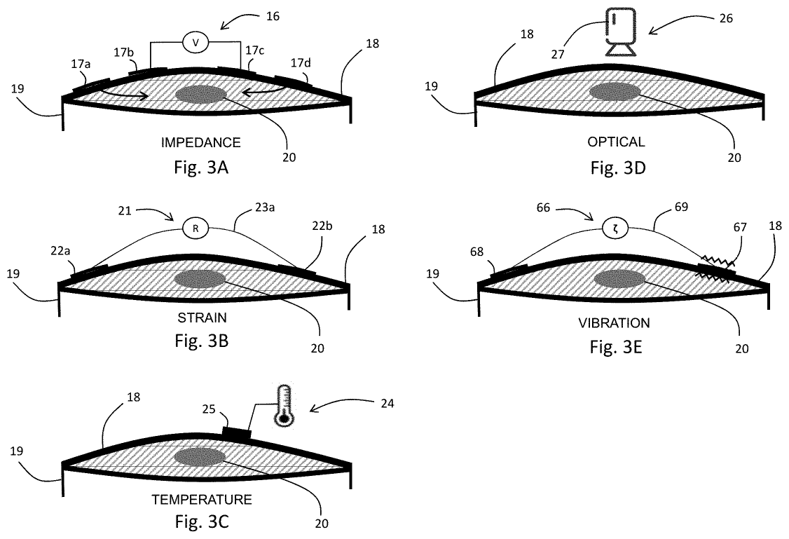

FIGS. 3A-E indicates how sensors 16, 21, 24, 26, 66 incorporated in the system shown in FIG. 2 can measure signals that characterize time-dependent swelling (e.g., that caused by lymphedema) in underlying tissue. Referring to FIG. 3A, an impedance sensor 16 features electrodes 17a-d that connects to a layer of skin 18 covering a portion of tissue 19 that includes a region of fluid build-up 20. The electrodes 17a-d connect to impedance circuitry (not shown in the figure) and sense signals that, once processed, determine the electrical impedance of tissue underneath them. More specifically, for the impedance measurement, the outer electrodes 17a, 17d, called ‘drive electrodes,’ inject a high-frequency (typically between 20-100 kHz), low-amperage (typically between 10-1000 μA) electrical current through the skin 18 and into the portion of tissue 19. Resistance of the surrounding tissue—which is affected by the fluid build-up 20—impacts current flow. It is manifested by a voltage drop measured by a pair of inner electrodes 17b, 17c called ‘sense electrodes. This voltage drop is digitized by an analog-to-digital converter in the impedance system to yield a time-dependent impedance waveform. Analysis of changes in this waveform can indicate the resistance change and ultimately the degree of fluid-build-up 20 in the tissue portion 19.

Similarly, as shown in FIG. 3B, the system in FIG. 2 can also include a sensor 21 that measures the strain induced by the fluid build-up 20 in the portion of tissue 19. In this embodiment, sensor 21 is typically secured to the skin 18 with adhesive elements 22a,b, e.g., an adhesive electrode, such as the drive 17a,d or sense 17b,c electrodes in FIG. 3A. The adhesive elements 22a,b connect to opposing distal ends of a conductor 23a. As the skin 18 bows due to underlying fluid build-up 20, the conductor 23a is stretched, causing its resistance to change. This means the conductor 23a functions as a variable resistor characterized by a resistance value that changes with strain, as such, swelling of the underlying tissue. The conductor 23a can thus be used in well-known circuits—such as a Wheatstone Bridge or a circuit featuring either serial or parallel resistors—that apply a constant voltage to contacts on the circuit and allow measurement of a voltage drop induced by the variable resistor (i.e., the stretched conductor 23a) to determine the degree of mechanical strain. The conductor 23a, for example, can be a patterned metal film (e.g., a thin metal layer deposited in a serpentine pattern) or wire, typically deposited on a stretchable, flexible substrate (e.g., a polymeric film).

Alternatively, in place of or in addition to a variable resistor, a sensor 21 that measures strain can feature a variable capacitor. Here, the conductor 23a is replaced by a capacitor, which is typically a dielectric medium (e.g., plastic, glass, or polymeric material) covered on two opposing sides by thin, electrically isolated metal films. As the capacitor is stretched by swelling in the tissue, the thickness of the dielectric medium is reduced, and the metal films are drawn closer together. This reduces the capacitance of the system. A circuit that features this variable capacitor in series with a second capacitor having a known, fixed value can yield a voltage drop when biased. Measuring the voltage drop indicates the degree of strain in the system and, in turn, the degree of swelling in the underlying tissue.

As described above, in place or in addition to the variable resistor or capacitor described above, a sensor 21 may include a piezoelectric material that generates a voltage that varies with the degree that the conductor 23a is stretched.

Similarly, as indicated in FIG. 3C, a temperature sensor 24 can measure swelling in the portion of tissue 19 caused by fluid build-up 20. The temperature sensor 24 typically features a temperature-sensitive element 25 (e.g., a thermocouple, temperature-sensitive resistor, or thermopile) adhered to the surface of the skin 18. Temperature measurements are predicated on the idea that swelling will cause the temperature of the portion of tissue 19 and the overlying skin to increase gradually. As shown in FIG. 3D, the system can also include an optical system 26, featuring an optical element 27 such as a camera (e.g., CCD camera) or combined light source, e.g., a light-emitting diode (herein “LED”) or laser, and photodetector (e.g., photodiode) to detect swelling as described above. For example, swelling may manifest as a change in coloration of the skin 18. A camera can capture an image of the skin to detect this condition. In related embodiments, the combined light source and photodetector can measure the optical absorbance and/or reflectance of the skin and use these parameters to estimate the degree of swelling.

Alternatively, as shown in FIG. 3E, a sensor 66 that senses mechanical vibrations that pass through the skin 18 to the portion of tissue 19 enclosing fluid build-up 20 can measure time-dependent changes in these materials' composition, thus be used to estimate the degree of lymphedema. Here, a vibrating element (e.g., piezoelectric crystal) 67 generates one or more signals characterized by known acoustic frequencies that travel through the portion of tissue 19 to the area of fluid build-up. A vibration sensor (e.g., piezoelectric crystal) 68 connected to the vibrating element 67 through a wire 69 measures these vibrations. The emitted and received vibrating signals are compared by a processor in the system to estimate parameters such as phase shift, damping ratio, and stiffness at one or multiple frequencies of vibration to assess changes in the composition of the portion of tissue due to the fluid build-up 20.

Sensors are other than those shown in FIGS. 3A-E can be incorporated in the system of the invention (or used in addition to the system) to estimate the degree of lymphedema. For example, as indicated in FIGS. 15A-B, a simple camera 103 incorporated into a mobile phone, or visual inspection (indicated by the eye 105) can image the semi-permanent tattoo 101a printed on the arm 102a of the patient 85. Swelling causes the separation between components (in this case, vertical lines) of the semi-permanent tattoo 101a in an arm 102a unaffected by lymphedema to gradually increase, as indicated by the tattoo 101a and arm 102b in the right-hand side of the figure. Such a change in separation of the components, once detected with camera 103 or through visual inspection, can be used to estimate the degree of lymphedema. Here, ‘detection’ can be made by processing the camera-generated image with an image-processing algorithm, e.g., one that analyzes pixels in the image to determine the separation of the components. Alternatively, the image-processing algorithm may incorporate more sophisticated techniques based on artificial intelligence or machine learning. The algorithm, for example, may be included in a software application running on a conventional ‘smartphone,’ thereby allowing the patient to diagnose the degree of lymphedema in the home environment without the need for a remote clinician. In related embodiments, the separation between the components can simply be manually measured with a conventional ruler 107.

### 2. CLINICAL RESULTS

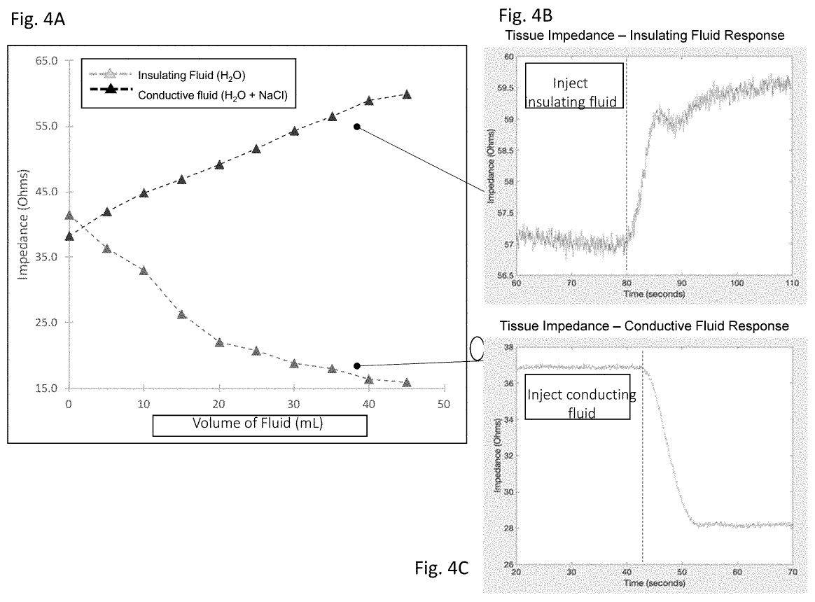

Referring to FIGS. 4A-C, swelling and electrical impedance's ability to measure it can be simulated with a benchtop rig. Here, a ‘phantom’ system was developed using a 3D-printed box filled with a mixture of agar (to provide a density similar to human tissue) and sponges (to absorb excess fluid). These components were covered with a layer of synthetic skin having electrical and mechanical properties matched to human skin (purchased from Syndaver Inc.; www.syndaver.com). An impedance sensor featuring two sense and two drive electrodes, similar to that shown in FIG. 3A, was then attached to the synthetic skin. The resultant system had a baseline impedance of about 37 Ohms. Once it was fully fabricated and the impedance sensor attached to its surface, a calibrated syringe was used to systematically inject both insulating (deionized water) and conductive (saline) solutions into the phantom. The impedance sensor then measured the resulting DC impedance signals.

As shown in FIG. 4A, as insulating deionized water is injected into the phantom, its overall impedance as measured by the impedance sensor systematically decreases. Likewise, injecting conducting saline into the phantom gradually increases the impedance. FIGS. 4B and 4C show, respectively, the time-dependent increase and decrease in the impedance signal, as measured by the impedance sensor. Dashed lines in each figure indicate when a small bolus (2 ml) of fluid is injected into the phantom. As indicated by the rapid change in DC signal level, the response time of the sensor to injected fluids is on the order of a few minutes; this is significantly faster than times scales associated with conditions such as lymphedema, which typically manifests over months.

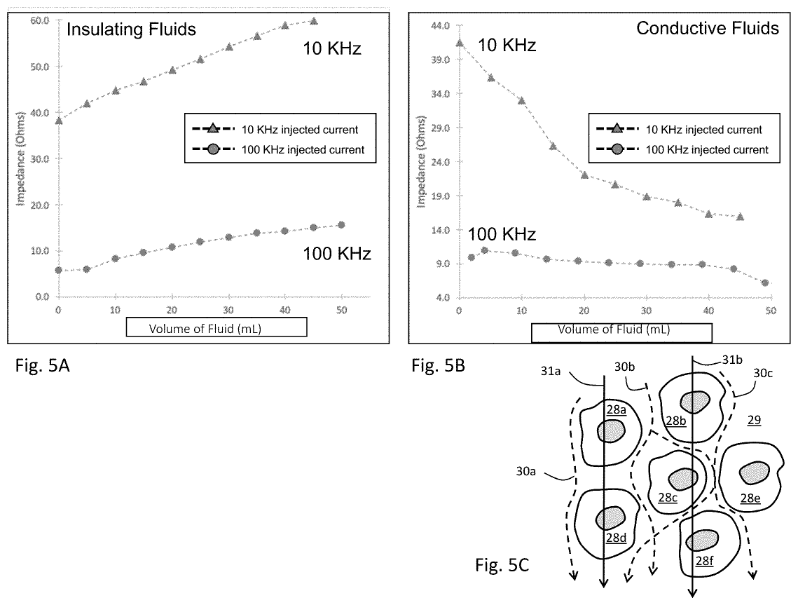

Referring to FIGS. 5A-C, the same phantom and impedance system used to generate data for FIGS. 4A-C was used to estimate the impact of the frequency of the injected current on signal magnitude. Here, using the same approach as described above, the frequency of the injected current was toggled between 10-100 KHz for both insulating deionized water (FIG. 5A) and conductive saline (FIG. 5B). As is clear from these figures, the change in impedance with a volume of injected fluid is more pronounced when measured at relatively low frequencies (e.g., 10 KHz) than higher frequencies (100 KHz). FIG. 5C shows a graphical representation of this phenomena that includes a series of cells 28a-f immersed in a fluid medium 29. Both the cells 28a-f and fluid medium 29 impact the impedance measurement made by the impedance sensor, as indicated in FIGS. 4A-C and 5A-B. However, as indicated by dashed lines 30a-c, relatively low frequencies of injected current pass around the cells 28a-f. Here, the capacitance of the cells' membranes prohibits current from passing through them. This indicates relatively low frequencies of injected current are likely to sample—and thus be more sensitive to—the extra-cellular fluids. These are likely the fluids that induce swelling. In contrast, the low-frequency measurement should be less sensitive to the intracellular fluid, as this current does not pass through the cells 28a-f. Conversely, relatively high frequencies of injected current 31a-b can overcome the inherent capacitance of a typical cell membrane, as pass through both the cells 28a-f and fluid medium 29. Since both, these components contribute to the impedance signal, but only the extra-cellular fluids are increased by swelling, the relatively high-frequency current is less sensitive to a swelling-induced signal change.

### 3. MECHANICAL AND ELECTRICAL COMPONENTS

FIG. 6A shows the schematic drawing of the system 6 as previously described in FIG. 2, consisting of: the hand-held component 9, the pair of disposable body-worn electrode patches 7a,b that adhere to the region of interest, the cable 8 that receives the bio-electric output signals from the two electrodes, and the stretchable cable 11 that sends the bio-electric signals from the cable 8 to the hand-held component 9.

FIGS. 6B, and 6C show, respectively, the schematic drawing of the disposable electrode patch 7a,b when viewed from the top-down perspective, and the bottom-up perspective. The body 32 of the patch 7a,b has three openings 33a-c that during use align with markings 34a-c (e.g. semi-permanent tattoos) on the patient to help guide the placement of the electrode patch on their skin. The electrodes 35a,b are on each patch 7a,b are sense and drive electrodes, as described above. They attach to the bottom surface 36 of the patch 7a,b, which also includes an adhesive film to adhere it to the patient's skin. FIG. 6D shows the placement 37 of system 6 onto the patient's body 10 using alignment with markings 34a-c and 34a-c′. FIG. 6E shows the fully placed system 6.

FIGS. 7A and 7B show, respectively, an image and photograph of a circuit board 38 used within the system described above. The figure shows the circuit board 38 is a 4-layer fiberglass/metal structure that includes metal pads soldered to, among other components, an analog-to-digital converter 39, accelerometer 40, operational amplifiers 41a-f, and power regulators 42a,b. More specifically, operational amplifiers 41a-d make up analog high and low-pass filters, and operational amplifiers 41e-f and power regulators 42a,b collectively regulate power levels for the various components in the circuit board 38. The accelerometer 40 measures the motion of the circuit board 38 and, in doing this, any part of the patient's body it is attached to. The analog-to-digital converter 39 digitizes analog impedance waveforms after being filtered and converts them into digital waveforms with 16-bit resolution and a maximum digitization rate of 200 Ksamples/second.

The circuit board 38 additionally includes sets of metal-plated holes that support a 4-pin connector 43, two 6-pin connectors 44, 45, and a 3-pin connector 46. More specifically, connector 43 connects directly to the pressure transducer, where it receives a common ground signal and analog impedance waveforms. These waveforms are filtered and digitized, as described in more detail below. Through connector 46 the circuit board receives power (+5V, +3.3V, and ground) from an external power supply, e.g., a battery or power supply located in the arm-worn housing. These power levels may be different in other embodiments of the invention. Digital signals and a corresponding ground from the analog-to-digital converter 39 are terminated at connector 45; they leave the circuit board 38 at this point, e.g., through cable segment 8 shown in FIG. 2. Connector 44 is used primarily for testing and debugging purposes and allows analog impedance signals, once they pass through analog high and low-pass filters, to be measured with an external device such as an oscilloscope.

The circuit board 38 typically connects to the electronics module through a serial interface (e.g., SPI, I2C), which includes components for processing, storing, and transmitting data digitized by the analog-to-digital converter 39. For example, the electronics module typically includes a microprocessor, microcontroller, or similar integrated circuit. It can additionally provide analog and digital circuitry for other measurements, such as those indicated by the sensors shown in FIGS. 3B-D. In embodiments, the microprocessor or microcontroller thereon can operate computer code to process impedance and other time-dependent waveforms to determine vital signs (e.g., HR, and other parameters, such as respiration rate, SpO2, stroke volume, cardiac output, and fluids) and associated parameters (e.g., wedge pressure, central venous pressure, blood volume, fluid volume, and pulmonary arterial pressure). “Processing” by the microprocessor in this way, as used herein, means using computer code or a comparable approach to digitally filter (e.g., with a high-pass, low-pass, and/or band-pass filter), transform (e.g., using fast Fourier Transforms, continuous wavelet transforms, and/or discrete wavelet transforms), mathematically manipulate, and generally process and analyze the waveforms and parameters and constructs derived therefrom with algorithms known in the art. Examples of such algorithms include those described in the following issued U.S. Patents, the contents of which are incorporated herein by reference: U.S. Pat. No. 11,129,537; 11,123,020; 11,116,410; 11,039,751; and 10,314,496.

In related embodiments, the electronics module can include both flash memory and random-access memory for storing time-dependent waveforms and numerical values, either before or after processing by the microprocessor. In still other embodiments, the circuit board can include NFC, Bluetooth®, Bluetooth Low Energy®, or Wi-Fi transceivers for both transmitting and receiving information.

FIGS. 8A-C shows another embodiment of the invention featuring a single electrode patch 47 that includes four distinct electrodes: sense the electrodes 48a,b and drives the electrodes 49a,b. The electrode patch 47 is shaped as an annular ring with an opening 50 in its center; through the opening 50, both an optical system 62 and/or camera (not shown in the figure) can image a patient's skin 51, as described in more detail below. The annular ring's bottom surface supports both the sense 48a,b and drives 49a,b electrodes surrounding the opening 50. Importantly, electrode patch 47 features an alignment feature, which, as shown in FIG. 8A, is a notch 52 cut into the annular ring. A primary purpose of notch 52 is to align with a corresponding alignment feature 53 that is part of a semi-permanent tattoo 54, which is printed on a patient's skin 51 prior to measurement. Here, the semi-permanent tattoo 54 features an annular ring shape with a diameter that is slightly less than that of the inner diameter of the annular ring-shaped electrode patch 47. The presence of these ‘concentric circles’—one corresponding to the electrode patch 47 and the other to the semi-permanent tattoo 54—allows for repeatable alignment of the patch 47, as described in more detail below.

The semi-permanent tattoo 54 is typically inscribed on the patient's skin 51 in a region above tissue inflicted with lymphedema. During use (typically, e.g., on a weekly basis) the electrode patch 47 is applied to the patient's skin 51 so that it is opening 50 is concentric with the circular semi-permanent tattoo 54, and its notch 52 is positioned directly proximal to the alignment feature 53 within the tattoo 54.

As shown in FIG. 8A, and similar to the embodiments described herein, the electrode patch 47 features strain gauges 55a-d, which in this case are metal traces patterned in a serpentine manner across the width of the annular ring. The resistance of the metal traces changes as they are strained by lymphedema-induced swelling in the underlying tissue. Each strain gauge—which effectively forms a variable resistor—connects to a conventional circuit, such as a Wheatstone Bridge, that generates a measurable voltage impacted by the strain gauges' variable resistance. To accommodate strain, the electrode patch 47 is typically composed of a stretchable material, similar, for example, to Tegaderm® manufactured by 3M (https://www.3 m.com/3M/en_US/p/c/b/tegaderm/i/health-care/medical/). The metal traces in the strain gauges 55a-d are terminated on each end with a pair of electrical contacts 56a-d; these are oriented to mate (i.e., come in electrical contact) with matched electrical contacts 58a-d patterned on a bottom surface 59 of a circular, battery-powered circuit board 57 that forms the primary component of a device 65 that measures lymphedema. When they touch, the electrical contacts 58a-d, 56a-d connect the strain gauges 55a-d (i.e., variable resistors) to individual Wheatstone Bridge circuits within the circuit board 57. Additionally, circuit board 57 features the first set of electrical contacts 60a,b for the sense electrodes 48a,b, and a second set of electrical contacts 61a,b for the drive electrodes 49a,b. The electrical contacts 60a,b, 61a,b are typically thin metal films that, during use, electrically connect to a conductive rivet (not shown in the figure) inserted into hydrogel electrodes 48a,b, 49a,b. During a measurement, the contacts 61a,b transmit alternating current (herein “AC”) into the patient's skin through the drive electrodes 49a,b while the contacts 60a,b port corresponding bioelectric signals to ECG and impedance circuits on the circuit board

57. For impedance measurements, the corresponding circuit typically injects alternating AC into the skin, wherein the frequency of the current typically varies from about 5 KHz to 500 KHz; in some embodiments, the circuit rapidly ‘sweeps’ the AC frequencies between these ranges.

Technically, the impedance measured with the circuit is a complex term, wherein electrical resistance encountered by the electrical current represents the real component of the impedance, and reactance encountered by the current represents the imaginary component of the impedance. More specifically to this particular measurement, impedance (the real component of the signal) is typically impacted by the volume of lymphedema-induced fluid, whereas reactance (the imaginary component of the signal) is typically impacted by the electrostatic storage of charge (i.e., capacitance) caused by the fluid.

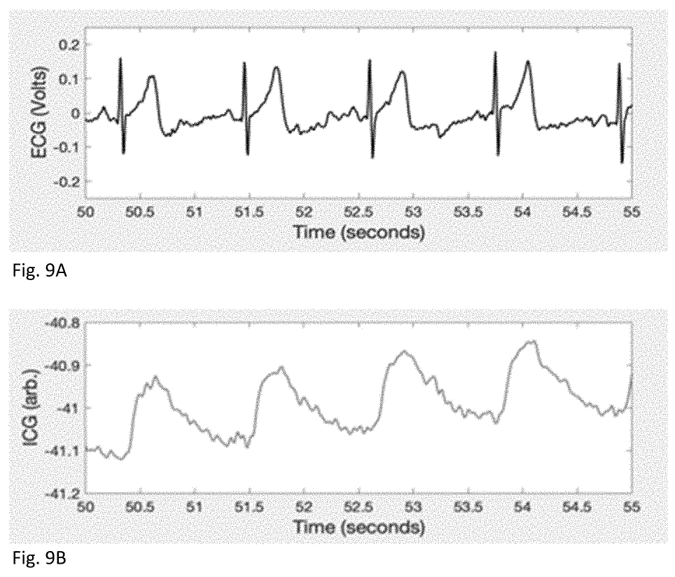

On a more practical level, the above-described circuits process the bioelectric signals (e.g., filter and amplify them) to generate, respectively, analog time-dependent ECG, bioimpedance, and bio-reactance waveforms. These are then digitized with an analog-to-digital converter to yield digital waveforms suitable for follow-on processing. FIGS. 9A and 9B, for example, show digital ECG and bioimpedance waveforms featuring heartbeat-induced pulses;

FIGS. 4B and 4C show time-dependent impedance waveforms characterized by a baseline that changes with underlying fluid. A microprocessor operating on the circuit board 57 runs computer code that uses algorithms to process these types of waveforms, extract any fiducial features (e.g., signal levels of the impedance and reactance waveforms, QRS complexes from the ECG waveforms), and then processes these to estimate the progression of lymphedema and other physiological conditions (e.g. variable of RR intervals extracted from the QRS complexes) in the patient that might indicate other disease states (e.g. arrhythmias).

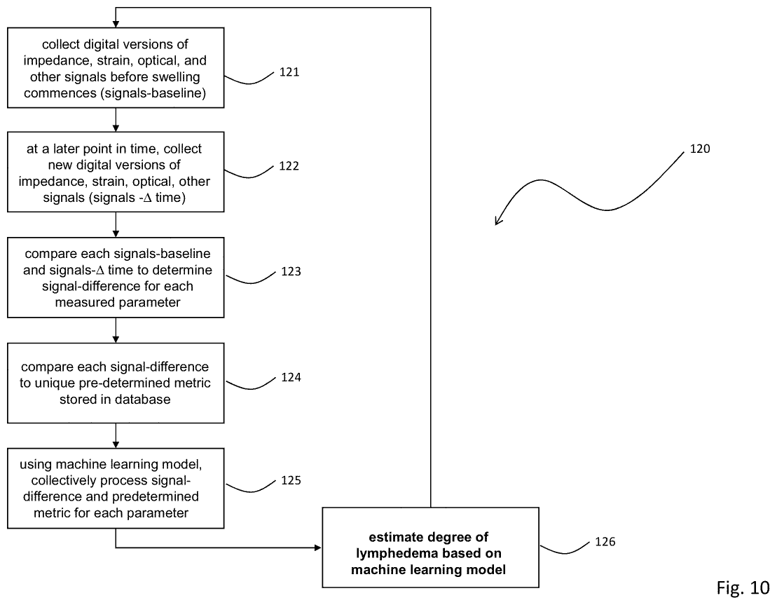

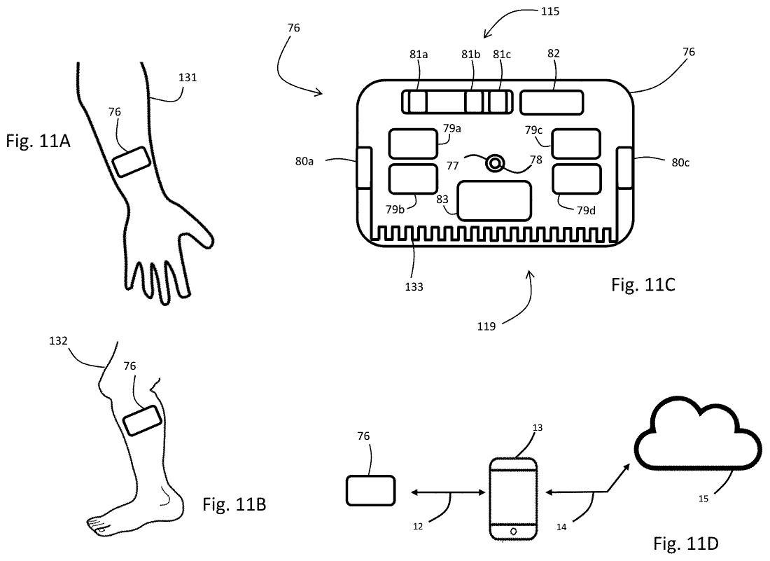

Additionally, the circuit board 57 includes an optical sensor 62 that, in turn, features a light source 63 and a photodetector 64 operating in a reflection-mode geometry to determine optical properties (e.g., coloration, texture) of the patient's skin 51. During use, light source 63 irradiates a region of the patient's skin 51 disposed above the area of lymphedema. Radiation reflecting off the region will be impacted by conditions such as redness and mottling of the skin 51; the photodetector 64 senses the reflected radiation and generates a signal, which is then filtered and amplified by the corresponding circuitry on the circuit board 57 to generate a time-dependent ‘optical’ waveform. An algorithm (e.g., that shown in FIG. 10) then analyzes the optical waveform to estimate changes in the skin coloration that may indicate the progression of lymphedema. In embodiments, the light source 63 may be a collection or array of light sources (typically light-emitting diodes or laser diodes), each emitting radiation at a different wavelength. Alternatively, the light source 63 may be a ‘white’ light source (e.g., a multi-wavelength LED or tungsten lamp) that emits a collection of wavelengths throughout the visible, infrared and ultraviolet spectra. The photodetector 64 is typically a single photodiode or an array of photodiodes. Alternatively, it can be an imaging system (e.g., a CCD camera) configured to collect a spatial image of the underlying skin.

During use, the electrode patch 47 is applied to the patient's skin 51 so that the opening 50 and the annular ring are concentric with the annular-shaped semi-permanent tattoo 54, and the patch's alignment feature 52 is proximal to the tattoo's alignment feature 53. If swelling is present, it may be necessary to stretch the patch 47 so that the tattoo 54 is entirely visible. This stretching, as described above, will induce strain in the strain gauges 55a-d. The device 65 featuring the above-described sensors then snaps into the electrode patch 47 so that electrical contacts 60a,b and 61a,b in electrical contact with an internal impedance circuit align with the different sets of sense 48a,b and drive 49a,b electrodes, and electrical contacts 58a-d align with the contacts 56a-d associated with the strain gauges. Note that all electrical contacts must be large enough to accommodate any stretching of the patch 47. With this process, the electrode patch 47 is consistently placed about the region of lymphedema, allowing repeatable measurements to be made using various circuits within the circuit board 57, as described above.

Typically, the electrode patch is composed of a foam substrate with an adhesive layer on its bottom surface. The sense 48a,b and drive 49a,b electrodes are typically made from a hydrogel material that is typically adhesive, electrically conductive and features an electrical impedance matched to the patient's skin. Electrical traces and contacts are typically composed of conductive materials, such as metal films or conductive ink. Electrodes may also be dry electrodes made of metals (e.g., tin, silver, sintered Ag/AgCl, gold, platinum, and stainless steel) or polymers (e.g., EDPM rubber with additives).

In related embodiments, a semi-permanent tattoo similar to that shown in FIG. 8A can be imaged (e.g., by taking a photograph of it with a conventional mobile phone) to indicate the degree of lymphedema-induced swelling. For example, the tattoo may include an annular ring and an alignment marker for the electrode patch, as shown in the figure, and additionally a small dot in its center. The distance between the dot and the perimeter of the annular ring may indicate the degree of swelling. During use, the patient may use their mobile phone and a customized software application to take a photo of the semi-permanent tattoo before the electrode patch is applied. The software application transmits the photograph to a cloud-based system, where image-processing algorithms (e.g., those using artificial intelligence or machine learning) can evaluate it to estimate separation between features in the tattoo and, from this, the degree of swelling. In embodiments, the semi-permanent tattoo may include other features (e.g., linear segments with well-defined lengths, dots with well-defined separations) that are known to the image-processing algorithms as a way to improve the estimation of swelling. Results from the image-processing algorithms and algorithms that process sensor-generated waveforms may be combined to enhance the determination of lymphedema. In other embodiments, both the electrode patch and the semi-permanent tattoo can have other shapes, such as square, rectangular, oval, etc.

In a related embodiment, circuits used in the above-described sensors can be temporarily printed on the patient's body using conductive ink or electroconductive paint. Such a circuit may interface with an onboard microelectronic chip and be configured with sensors such as a strain gauge to determine the expansion of the limb and electrodes to assess limb fluid content using bioimpedance.

FIG. 10 shows a typical algorithm 120 based on machine learning that estimates the degree of lymphedema by processing information generated as described above. As shown, the algorithm 120 includes the following steps:
STEP 121: collect digital versions of impedance, strain, optical, and other signals before swelling commences (the resulting value is referred to herein as “signals-baseline”)
STEP 122: at a later point in time, collect new digital versions of impedance, strain, optical, other signals (the resulting value is referred to herein as “signals −Δ time”)
STEP 123: compare each signals-baseline and signals-A time to determine a new value representing a mathematical difference for each measured parameter (the resulting value is referred to herein as “signal-difference”)
STEP 124: compare each signal-difference to unique pre-determined metric stored in database
STEP 125: using a machine learning model, collectively process signal-difference and predetermined metric for each parameter
STEP 126: estimate degree of lymphedema based on the machine learning model

Steps 122-126 of this process are typically repeated on a somewhat periodic basis, e.g. every week, in order to help the patient keep their lymphedema under control. (Note: step 121, i.e. collecting the signals-baseline value, is only collected when the sensor is initially deployed.)

### 4. ALTERNATIVE EMBODIMENTS

In other embodiments, the disposable electrodes (typically composed of a conductive hydrogel that contacts the patient and an adhesive foam backing) described above may be replaced by small ‘epidermal electrodes’ composed of conductive polymers that are printed directly on the patient's skin. Such epidermal electrodes feature several advantages over their disposable counterparts. They ensure consistent placement, reduce the burden on the patient to periodically apply disposable electrodes, and reduce skin irritation caused by the adhesive and hydrogel components within disposable electrodes. Epidermal electrodes provide a conductive interface with the patient's body through their skin. A conductive probe contacting them can detect electrophysiological signals that, once processed, yield time-dependent ECG and bioimpedance waveforms; as described above, further processing of these waveforms indicates early signs of lymphedema progression.

The conductive material used in epidermal electrodes may be drawn directly on the patient's skin with conductive ink. Alternatively, the electrode may be inkjet-printed onto standard tattoo paper and then transferred onto the skin in a similar fashion to a conventional press-on temporary tattoo. Such a system is referred to as the “electrode tattoo” in the following paper, the contents of which are incorporated herein by reference: Ferrari et al., 2020, the Institute of Solid State Physics, Graz, Austria. In this application, a system similar to that shown in FIG. 8B-C contacts the epidermal tattoo using conductive metal contacts (similar to components 60a,b, 61a,b, 58a-d) that protrude outward. To make a measurement, the system, for example, is manually pressed against the patient's skin so that the protruding metal contacts come into contact with the epidermal electrodes. Signals are then collected and processed in the same manner as that done using standard hydrogel electrodes.

FIGS. 11A-D show another alternative embodiment of the invention featuring a patch 76 that includes the sensors previously described in FIGS. 3A-E. As shown, the patch 76 is configured to be worn on a patient's extremity, e.g., a limb such as an arm 131 or a leg 132, but can also be worn on other parts of the body. FIG. 11C shows a top view of the patch 76 and its alignment feature 77. The alignment feature 77, for example, can be one of an opening, optically transparent area, notch, or cut-out area that during use is disposed above a marking 78. The marking 78, for example, can be a permanent or semi-permanent marking, such as a tattoo. The tattoo can be a conventional tattoo, or alternatively a tattoo that has a limited lifetime, e.g., about one year. The patch includes four electrodes 79a-d that connect to an ECG/bioimpedance circuit: two are current-injecting electrodes 79a,d and a two are sensing electrodes 79b,c. During a measurement, the current-injecting electrodes 79a,d typically inject high-frequency (e.g., 100 kHz), low-amperage (e.g., 4 mA) current into the portion of the patient impacted by lymphedema; the current and amount of underlying fluid or adipose tissue impacts the bio-electric signals sensed by the sensing electrodes 79b,c. Additionally, the patch 76 also includes a strain gauge 119 featuring a conductive trace 133 terminated at its distal ends with conductive adhesive elements 80a, 80c. As the limb swells due to lymphatic fluid buildup, the conductive trace 133 is stretched, causing its resistance to change; this means the strain gauge 119 functions as a variable resistor characterized by a resistance value that changes with strain and, as such, swelling of the underlying tissue. Typically to form the strain gauge 119 the conductor 133 is incorporated as a variable resistor that is used in well-known circuits—such as a Wheatstone Bridge or a circuit featuring either serial or parallel resistors—that apply a constant voltage to the conductive adhesive elements 80a, 80c. A voltage drop across the circuit induced by the variable resistor (i.e., the stretched conductor 133) indicates the degree of mechanical strain. The conductor 133, for example, can be a patterned metal film (e.g., a thin metal layer deposited in a serpentine pattern) or wire, typically deposited on a stretchable, flexible substrate (e.g., a polymeric film).

The patch 76 may also contain an optical system 115 featuring a light source 81a, such as an LED, and two photodetectors, 81b-c. In one embodiment, the combined light source 81a and photodetectors 81b,c measure the optical absorbance and/or reflectance of the skin and use these parameters to estimate the degree of swelling. In embodiments, for example, the optical system 115 is similar to that used in near-infrared spectroscopy (herein “NIRS”). With NIRS or any optical system used in the invention, the light source 81a emits optical radiation at multiple frequencies within the visible, ultraviolet, and/or infrared spectrum directly into the tissue portion. As the light travels through the tissue, photons are absorbed, reflected, and dispersed. Some photons will arrive to the photodetectors 81b,c and generate a signal (typically a time-dependent one) called a photoplethysmogram (herein “PPG”). Analysis of the properties of one or more PPGs can yield the composition of the underlying tissue as well as the concentration of certain molecules (e.g., Hb, HbO2, H2O, fat) therein. The concentration of these molecules is calculated according to the Beer-Lambert Law, where:

Absorbance (A)=Molar Attenuation Coefficient (ε)*Optical Path Length (L)*Concentration of Molecule (c)

In a typical embodiment, optical wavelengths in the 690-905 nm range can be used to measure Hb, HbO2, and melanin; longer wavelengths can be used to measure human fat content (e.g., 915 nm, 1210 nm, 1720 nm). Changes in oxygen perfusion and adipose tissue buildup relate to the progression of lymphedema due to swelling, inflammation, or necrosis. Melanin can be used to correct the absorption signals for patients of different skin tones.

The patch 76 also includes a temperature sensor 82 that measures temperature changes due to swelling in the portion of tissue underneath the patch. The temperature sensor 82 typically features a temperature-sensitive element (e.g., a thermocouple, temperature-sensitive resistor, thermopile, or thermal flow sensor) adhered to the surface of the skin. This particular measurement is predicated on the idea that swelling will cause the temperature of the portion of tissue and overlying skin to gradually increase. Bioelectric signals collected from the sensors 115, 119, 82 are then processed by a processor element 83, such as a microprocessor or microcontroller. As shown in FIG. 11D, patch 76 can transmit information (as indicated by arrow 12) to a software application running on the patient's mobile phone 13, which in turn transmits the information (as indicated by arrow 14) to a software system residing in the cloud 15.

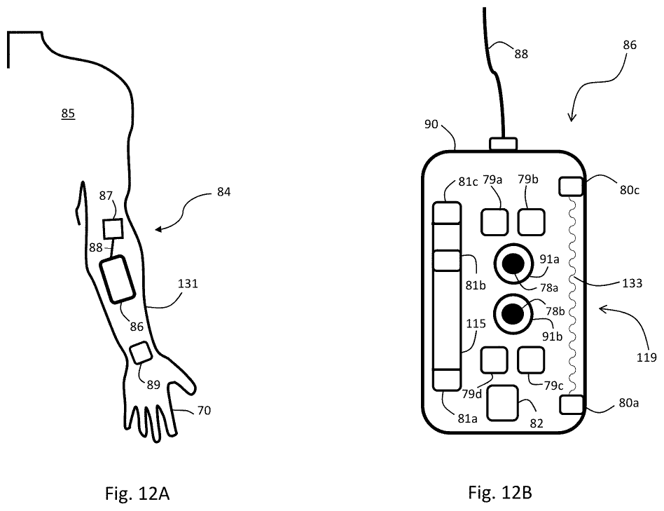

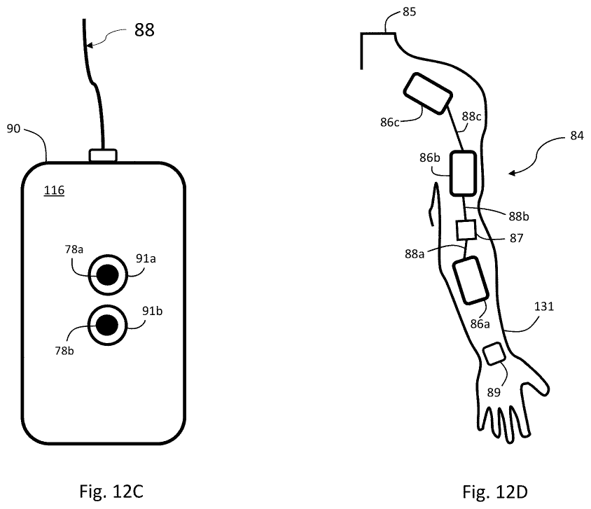

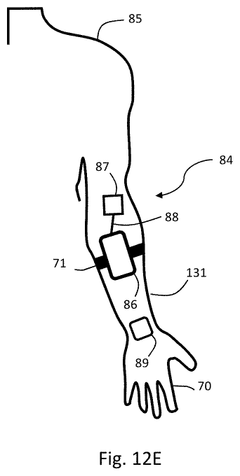

FIG. 12A-B shows an alternative embodiment of the invention featuring a sensor system 84 distributed along the patient's body featuring a patch 86 similar to that described above that delivers signals through a flexible cable 88 to a remote processor 87. Sensors within patch 86 include an optical sensor 115, a strain gauge 119, a temperature sensor 82, two drive electrodes 79a,b coupled two sensing electrodes 79c,d associated with an ECG/bioimpedance system. Each of these sensors transmits information to processor 87 through the flexible wire 88. The patch 86 is typically enclosed with a housing module 90 composed of flexible material (e.g., a polyurethane membrane, foam, or other plastic composition) and affixes to the skin of the affected limb (in this case, the patient's arm 131) using an adhesive layer 116. Alternately, as shown in FIG. 12E, the housing module 90 can attach to the patient's body through one or more straps 71, which typically wrap around the patient's arm 131 or finger 70. The bottom surface of the housing module 90 contains two alignment features 91a,b, each consisting of a transparent area or notch. During use is aligned to semi-permanent markings 78a,b printed on the patient's skin, as described previously. The alignment of these features allows for consistent device positioning. Alternately, the semi-permanent marking 78a,b may consist of a conductive ink tattoo. Here, the user positions patch 86 so that the alignment feature 91a,b aligns with the semi-permanent markings 78a,b; this component, in turn, conducts bioelectric signals through its conductive ink. In this way, the semi-permanent markings 78a,b serve as an electrode to supply input signals to the ECG/bioimpedance circuits. A housing module 90, similar to that described above, encloses the multiple sensor systems 79a-d, 82, 115, 119 as previously described. The remote processor 87 receives and analyzes signals from these sensors and wirelessly transmits the resulting information to a remote smartphone, further analyzing the information according to the algorithm described in FIG. 10. The processor 87 may affix to the patient's arm 131 using an adhesive 116 or strap 71. In an alternate embodiment, the processor 87 may be held within the subject's hand or placed within a pocket.

Alternatively, as shown in FIGS. 12C-D, the system can also include a multi-patch system 86a-c with three or more measurement sites to determine the lymphatic flow. Here, an injection site 89 represents a location where fluorescent dye (e.g., ICG) or another tracer (e.g., methylene blue) is injected into the patient using a needle, microneedle patch, or other method and preferentially absorbed into the patient's lymphatic vessels. The light source 81a typically emits optical radiation at multiple frequencies within the visible or infrared spectrum. The injected tracer partially absorbs the radiation based on the absorptive properties of the tracer (e.g., ICG at 780 nm; methylene blue at 665 nm). Some optical radiation may be emitted due to fluorescence (e.g., ICG at 830 nm). Photodetectors 81b,c measure the transmitted optical radiation and send the resultant signals to the processor 87, which determines the concentration of the tracer based on the magnitude of the measured signals. As the tracer travels throughout the patient's limb, its concentration is measured over time. Analysis of this time-dependent signal indicates the patient's rate of lymphatic transit. The multi-patch embodiment shown in FIGS. 12C-D enables quantification of the lymphatic transit rate through different segments of the limb, thereby enabling measurement of lymphatic function at various points along the affected extremity.

In place of dye molecules, the injected tracer can be metal-based (e.g., Magtrace® iron nanoparticles available from www.mammotome.com/products/magtrace/), or a hypertonic fluid (e.g., 2-23% NaCl saline solution). In this case, as the tracer/ions travel through the patient's limb, the signal is measured by the sense electrodes normally used in the ECG/bioimpedance systems described above. Processor 87 can determine the concentration of the tracer based on the measured signals.

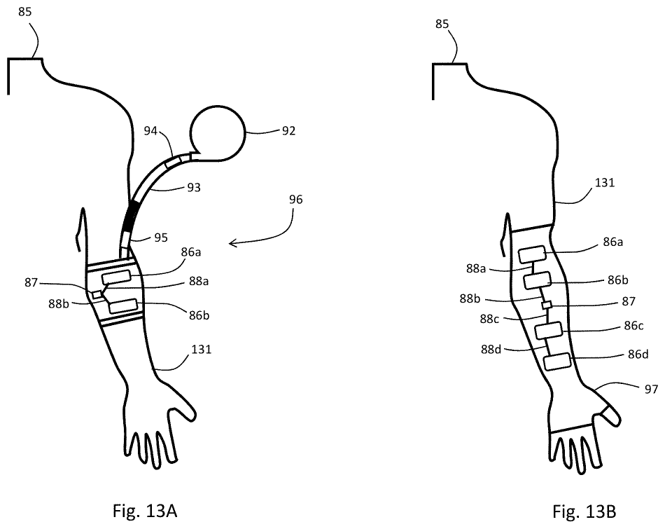

As shown in FIGS. 13A-B, in another alternate embodiment, a system 96 according to the invention includes electrical components similar to those described above, e.g. sensor-containing patches 86a,b and a separated processor 87 connected to each other through flexible cables 88a,b and woven into an inflatable cuff 96 (FIG. 13A) or sleeve 97 (FIG. 13B). Each patch 86a,b contains the same sensors as those shown in FIGS. 11C and 12B. The electrical components and internal parts of the sensors are enclosed within the fabric of the cuff 96 or sleeve 97, while the points of contact on the electrodes poke out of the fabric on its inner surface to contact the patient's skin. Here, the electrodes are typically reusable materials, such as conductive fabric, foam, or rubber. Referring to FIG. 13A, during use, an air pump 92 inflates the cuff 96. In doing so, it presses the sensors—including the strain sensor, optical sensor, temperature sensor, and electrodes that connect to the ECG/bioimpedance circuits—against the patient's body. Once the cuff 96 is fully inflated, its internal pressure is maintained, thus ensuring that each sensor contacts the patient's skin at constant pressure; this helps ensure consistent measurements. During use, the cuff 96 inflates through a pump 92 that pumps air through a rubber tube 93. The air then fills the cuff 96, causing it to expand and apply pressure on the skin. To measure the pressure within the cuff 76, the system includes a pressure gauge 94. The rubber tube 93 is detachable, so the user does not have to constantly hold it while using the cuff 96. It attaches and detaches from valve 95 which locks whenever tube 93 is detached so air cannot escape. Air can be let out if the user compresses valve 95 when tube 93 is not attached.

Referring to FIG. 13B, sleeve 97 is typically composed of a stretchable neoprene-type material, and can include multiple sensor-containing patches 86a-d, each of which connects to a central processor 87 through a flexible cable 88a-d. Each patch 86a-d includes the same types of sensors as those described above.

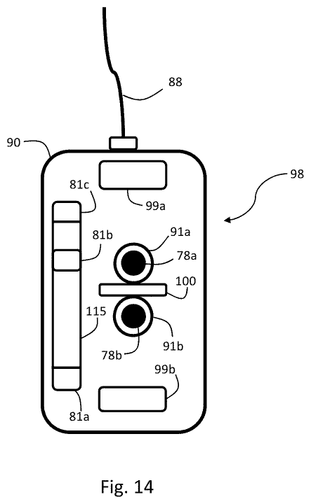

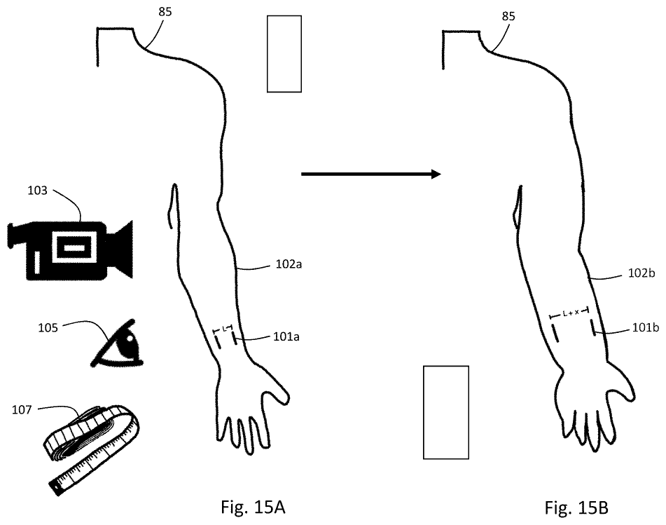

As shown in FIG. 14, another embodiment of the invention includes a patch 98 that includes an EMG electrode system in place of bioimpedance sensors. The patch includes optical sensor 115 featuring a light source 81a and two photodetectors 81b,c. The EMG electrode system consists of two electrode contact points 99a,b on opposite ends of a third contact serving as ground 100. The electrode contact points 99a,b and ground 100 detect electrical signals from underlying muscle nerves as the patient's muscles flex or extend. Patch 98 includes circuitry that measures the difference in electrical signal between the contact points 99a-b with respect to the signal measured from the ground 100 as a point of reference. Signals measured with these components will be impacted by lymphedema-induced changes fluids and adipose tissue. Such signals, once processed, can thus indicate the progression of lymphedema.

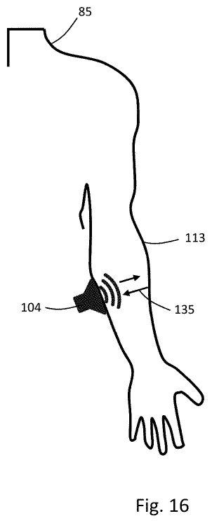

Referring to FIG. 16, in any of the above-described embodiments of the invention, the patch can include an ultrasound element 104 capable of generating and receiving ultrasound waves (20 KHz-20 MHz), indicated by arrows 135 in the figure. In embodiments, the ultrasound element 104 is a piezoelectric transducer. During a measurement, ultrasound waves 135 emitted from the ultrasound element 104 travel across a cross-section of a limb (such as the arm 113 shown in the figure), and are reflected back, where they are received by the ultrasound element 104. The signals are then digitized and analyzed by a processor. During lymphedema, the limb often accumulates fluid or fat. The processor interprets the reflected waves to determine the composition of the tissue (e.g., skin, fat, and fluid); further analysis can additionally determine the total thickness of the limb based on the type of tissue, the distance traveled by the ultrasound waves, the speed of sound, and the time required for an ‘echo’ to return. For example, as the limb swells, the distance between echoes increases, which can be used to calculate the degree of swelling.

These and other embodiments of the invention are deemed to be within the scope of the following claims.

What is claimed is:

1. A system for characterizing fluids in a tissue located in a portion of a patient, comprising:
an impedance system comprising at least one current-injecting electrode configured to inject an electrical current into the portion of the patient, and at least one signal-measuring electrode configured to measure an impedance signal affected by the injected electrical current and an amount of the fluids, wherein the system comprises an alignment feature that during use is aligned on the portion using a marking on the portion; and
a processing system configured to receive the impedance signal from the impedance system, or a signal determined therefrom, and then process it to determine a parameter related to the degree of fluids in the tissue.

2. The system of claim 1, wherein the alignment feature is one of an opening, optically transparent area, notch, or cut-out area configured to be disposed above the marking.

3. The system of claim 2, wherein the opening, optically transparent area, notch, or cut-out area is configured to allow the marking to be visualized when at least one of the current-injecting electrode and the signal-measuring electrode is attached to the portion.

4. The system of claim 1, wherein the alignment feature is a component configured to contact the patient proximal to the marking.

5. The system of claim 1, wherein the impedance system comprises two current-injecting electrodes and two signal-measuring electrodes.

6. The system of claim 5, wherein the impedance system comprises two electrode patches, with each electrode patch comprising one current-injecting electrode and one signal-measuring electrode.

7. The system of claim 6, further comprising an electrical cable connecting the two electrode patches.

8. The system of claim 7, wherein the electrical cable further comprises a conductor characterized by a resistance that changes with mechanical strain.

9. The system of claim 8, wherein the conductor is comprised by a strain gauge.

10. The system of claim 8, wherein the conductor is in electrical contact with a distance-measuring component that generates a signal related to a degree that the stretchable cable stretches.

11. The system of claim 10, wherein the distance-measuring component comprises a capacitor characterized by a capacitance value that changes with the degree that the stretchable cable stretches.

12. The system of claim 1, further comprising an optical system comprising a light source and a photodetector.

13. The system of claim 12, wherein the light source is configured to irradiate the tissue located in the portion of a patient.

14. The system of claim 13, wherein the photodetector is configured to detect radiation from the light source after it irradiates the tissue and, in response, generate a radiation-induced signal.

15. The system of claim 14, wherein the processing system is further configured to receive the radiation-induced signal, or a signal determined therefrom, and process it along with the impedance signal or a signal determined therefrom to determine the parameter related to the degree of fluids in the tissue in the portion of the patient.

16. The system of claim 1, further comprising an ECG system in electrical contact with the signal-measuring electrode and configured to measure an ECG waveform.

17. The system of claim 1, further comprising an EMG system in electrical contact with the signal-measuring electrode and configured to measure EMG signals from muscle neurons in the patient.

18. The system of claim 17, wherein the processing system is further configured to process the EMG signals to determine adipose tissue concentration corresponding to the patient.

19. A system for characterizing fluids in a tissue located in a portion of a patient, comprising:
an impedance system comprising at least one current-injecting electrode configured to inject an electrical current into the portion of the patient, and at least one signal-measuring electrode configured to measure an impedance signal affected by the electrical current and the fluids;
a distance-measuring system comprising a stretchable cable connected on one distal end to the at least one current-injecting electrode and at an opposing distal end to the at least one signal-measuring electrode, the distance-measuring system configured to generate an electrical signal that varies with a degree that the stretchable cable is stretched; and
a processing system configured to receive the impedance signal from the impedance system and the electrical signal from the distance-measuring system, or signals determined therefrom, and collectively process them to determine an index related to an amount of fluids in the tissue in the portion of the patient.

20. A system for characterizing fluids in a tissue located in a portion of a patient, comprising:
an impedance system comprising at least one current-injecting electrode configured to inject an electrical current into the portion of the patient, and at least one signal-measuring electrode configured to measure an impedance signal affected by the electrical current and the fluids;
a strain-measuring system comprising an electrical conductor coupled to the portion of the patient, the electrical conductor characterized by an electrical resistance signal that varies with strain imparted on the electrical conductor by the fluids; and
a processing system configured to receive the impedance signal from the impedance system and the electrical resistance signal from the strain-measuring system, or signals determined therefrom, and collectively process them to determine an index related to an amount of fluids in the tissue in the portion of the patient.
